(19) United States
(12) Patent Application Publication
(10) Pub. No.: US 2026/0256510 A1
(43) Pub. Date: Sep. 3, 2026

(54) METHODS AND DEVICES FOR ABLATION OF TISSUE

(71) Applicant: AtriCure, Inc., Mason, OH (US)

(72) Inventors: Kerry MONGILIO, Mason, OH (US); Robert KLEIN, Mason, OH (US); Christoph GILLUM, Mason, OH (US); Wanran MA, Mason, OH (US); Chandler KELLY, Mason, OH (US); Kenneth MILLER, Mason, OH (US); Josie FRIEDL, Mason, OH (US); Kurtis KLINGENBERG, Mason, OH (US); Geni RUPLEY, Mason, OH (US); Salvatore PRIVITERA, Mason, OH (US); Matthew WINKLER, Mason, OH (US); Randall J. LEE, Mason, OH (US); Grant SCHWIEBERT, Mason, OH (US); Jacob LUISI, Mason, OH (US)

(73) Assignee: AtriCure, Inc., Mason, OH (US)

(21) Appl. No.: 19/372,035

(22) Filed: Oct. 28, 2025

Related U.S. Application Data
(60) Provisional application No. 63/806,372, filed on May 15, 2025.
(60) Provisional application No. 63/792,522, filed on Apr. 22, 2025.
(60) Provisional application No. 63/765,390, filed on Feb. 28, 2025.

(51) Int. Cl. A61B 18/14; A61B 17/00; A61B 18/00

(52) U.S. Cl. CPC A61B 18/1445; A61B 2017/00566; A61B 2018/00083; A61B 2018/00351; A61B 2018/00577; A61B 2018/00839; A61B 2018/1465; A61B 2018/1467

(57) ABSTRACT

In some aspects, the techniques described herein relate to an ablation device, the device including: a handle; an elongated shaft extending from the handle along a longitudinal axis; a flexible electrode array at a distal end of the elongated shaft, wherein the flexible electrode array includes a plurality of electrodes and an insulative portion, wherein each of the plurality of electrodes extend longitudinally along the flexible electrode array, one or more slits extending transverse to the longitudinal axis, the one or more slits configured to increase the flexibility along a length of the flexible electrode array; and an end effector at the distal end of the elongated shaft, the end effector including a housing in which the flexible electrode array resides, wherein the housing is flexible and is configured to conform to a tissue of a patient.

### CROSS-REFERENCE TO RELATED APPLICATION

This application claims the benefit of U.S. Provisional Application No. 63/765,390 filed on February 28, 2025, U.S. Provisional Application No. 63/792,522 filed on April 22, 2025, and U.S. Provisional Application No. 63/806,372 filed on May 15, 2025, the content of each is incorporated herein by reference in their entirety.

### FIELD OF TECHNOLOGY

The present disclosure relates generally to the field of electrosurgical devices, and more specifically, ablation devices with a flexible electrode array and an end effector to deliver energy to tissue.

### BACKGROUND

One-sided ablation devices have benefits to ablate tissue from either extravascular or endovascular sides. However, current and historical one-sided radio-frequency ablation (RFA) devices have drawbacks. RFA devices typically operate in the 100-500kHz range, 10-100 VAC, and 10-100 seconds. Some RFA devices are monopolar, require anticoagulant, require active cooling, require a long ablation time, and/or have limited lesion depth. Some RFA devices may lead to excessive temperatures, either on the device back side (risk of damage to nearby tissue), on the tissue side (risk of tissue scorching and/or steam pops), and/or on the interior of the heart (risk of blood coagulation).

Pulsed field ablation (PFA) operates under different conditions, with pulses of high voltage & varying pulse widths. PFA has a lower thermal load than RFA due to the short durations; however, PFA has additional requirements (high voltage generators) and high voltage risks relative to RFA. PFA is also difficult to visualize as there is not significant thermal marking.

As a result, devices that can perform both improved RFA as well as PFA are of interest, to provide improved RFA performance as well as PFA capabilities.

Therefore, there remains a need for methods and devices that are capable of both RFA and PFA applications.

### SUMMARY

In some aspects, the techniques described herein relate to an ablation device, the device including: a handle; an elongated shaft extending from the handle along a longitudinal axis; a flexible electrode array at a distal end of the elongated shaft, wherein the flexible electrode array includes a plurality of electrodes and an insulative portion, wherein each of the plurality of electrodes extend longitudinally along the flexible electrode array, one or more slits extending transverse to the longitudinal axis, the one or more slits configured to increase the flexibility along a length of the flexible electrode array; and an end effector at the distal end of the elongated shaft, the end effector including a housing in which the flexible electrode array resides, wherein the housing is flexible and is configured to conform to a tissue of a patient.

In some aspects, the techniques described herein relate to an ablation device, wherein the one or more slits includes one or more pairs of slits extending laterally at opposite sides of the flexible electrode array.

In some aspects, the techniques described herein relate to an ablation device, wherein individual slits of the one or more pairs of slits are colinear to each other in a lateral direction transverse to the longitudinal axis.

In some aspects, the techniques described herein relate to an ablation device, wherein individual slits of the one or more pairs of slits are misaligned with each other in a lateral direction transverse to the longitudinal axis.

In some aspects, the techniques described herein relate to an ablation device, wherein the plurality of electrodes includes one or more ablation electrodes and one or more sensing electrodes.

In some aspects, the techniques described herein relate to an ablation device, wherein the flexible electrode array includes a center slit extending longitudinally through a center of the flexible electrode array.

In some aspects, the techniques described herein relate to an ablation device, wherein the flexible electrode array includes a plurality of arms, wherein the plurality of arms are collapsible within the end effector.

In some aspects, the techniques described herein relate to an ablation device, wherein the flexible electrode array further includes a tail including one or more electrical traces connected to the plurality of electrodes.

In some aspects, the techniques described herein relate to an ablation device, wherein the flexible electrode array includes a plurality of cutouts configured to bend the flexible electrode array.

In some aspects, the techniques described herein relate to an ablation device, further including a suction source coupled to the end effector, the suction source configured to apply suction to the end effector to draw tissue into the housing.

In some aspects, the techniques described herein relate to an ablation device, further including an energy source coupled to the plurality of electrodes, the energy source configured to provide energy to the plurality of electrodes.

In some aspects, the techniques described herein relate to an ablation device, further including a sensing source coupled to the plurality of electrodes, the sensing source configured to allow the electrodes to pace or sense electrical signals.

In some aspects, the techniques described herein relate to an ablation device, the device including: an elongated shaft along a longitudinal axis; a flexible electrode array at a distal end of the elongated shaft, wherein the flexible electrode array includes a plurality of electrodes and an insulative portion; and an end effector at the distal end of the elongated shaft, the end effector including a housing in which the flexible electrode array is anchored, wherein the housing is flexible to conform to a tissue of a patient.

In some aspects, the techniques described herein relate to an ablation device, wherein the end effector includes one or more cutouts.

In some aspects, the techniques described herein relate to an ablation device, further including a plurality of articulation bands attached to a distal tip of the end effector.

In some aspects, the techniques described herein relate to an ablation device, further including a plurality of heat stakes coupled to the end effector and configured to retain the flexible electrode array within the housing.

In some aspects, the techniques described herein relate to an ablation device, further including a plurality of notches within an inner surface of the housing.

In some aspects, the techniques described herein relate to an ablation device, wherein the end effector includes a channel extending along a length of the housing.

In some aspects, the techniques described herein relate to an ablation device, wherein the end effector includes one or more flexible ribs within the housing.

In some aspects, the techniques described herein relate to an ablation device, wherein the end effector includes one or more metallic stabilization elements within the housing.

### BRIEF DESCRIPTION OF THE DRAWINGS

FIG. 1A illustrates an example ablation device according to one variation of the present invention.

FIG. 1B illustrates close-up views of the end effector according to one variation of the present invention.

FIG. 1C illustrates one variation of the end effector comprising a pivot joint between the elongated shaft and the end effector.

FIGS. 2A and 2B illustrate variations of the flexible electrode array comprising a plurality of electrodes and a plurality of slits.

FIG. 3A illustrates the flexible electrode array within the end effector according to one variation of the present invention.

FIG. 3B illustrates cross-sectional diagrams of the flexible electrode array bending at various angles.

FIGS. 4A and 4B illustrate another variation of the flexible electrode array having one or more pacing and/or sensing electrodes.

FIGS. 5A and 5B illustrate another variation of the flexible electrode array having misaligned lateral slits.

FIG. 6 illustrates a variation of the flexible electrode array comprising one or more flexible arms each connected to electrical traces.

FIGS. 7A and 7B illustrate a variation of the flexible electrode array comprising two or more tails.

FIG. 8 illustrates a variation of the flexible electrode array comprising two distinct pads comprising distinct electrode areas.

FIG. 9 illustrates a variation of the flexible electrode array comprising outside electrodes having a pattern of block-shaped cutouts to increase flexibility in one plane.

FIG. 10A illustrates tissue partially folded when pulled into the end effector.

FIGS. 10B to 10E illustrate various configurations of energy application to the tissue via the electrodes.

FIG. 11A illustrates one variation of an end effector insert having one or more slits on the end effector.

FIGS. 11B and 11C illustrate the end effector bending with the flexible electrode array positioned within the end effector.

FIGS. 12A to 12C illustrate a variation of the end effector comprising a plurality of articulation bands.

FIGS. 13A and 13B illustrate a variation of the end effector comprising retention features for placing the flexible electrode array within the housing.

FIG. 14 illustrates a variation of the end effector comprising one or more compression notches on the inside of the housing.

FIG. 15 illustrates a cross-sectional view of one variation of the end effector and the flexible electrode array in which the end effector comprises one or more cutouts or flaps.

FIG. 16 illustrates a variation of the end effector comprising one or more cutouts and a channel that runs along the length of the end effector.

FIGS. 17A and 17B illustrate variations of an insert for use within the end effector.

FIGS. 18A to 18C illustrate one variation of the insert for use within the end effector comprising a hinge mechanism.

FIGS. 19A and 19B illustrate one variation of the insert for use within the end effector comprising a continuous gear.

FIG. 20 illustrates a variation of the flexible electrode array comprising a tail in a helical configuration within a vacuum lumen.

FIG. 21 illustrates a variation of the end effector having one or more external heat stakes.

FIGS. 22A to 22E illustrate variations of the flexible electrode array having creases.

FIG. 23A illustrates a handle with an integrated vacuum switch for use with the device.

FIG. 23B illustrates a closed configuration of the handle of FIG. 23A.

FIG. 23C illustrates a close-up view of the switch of FIG. 23A having a compression spring toggle.

FIG. 23D illustrates another variation of the handle comprising a rocker switch and a tensile spring.

FIG. 23E illustrates another variation of the handle comprising a clicking element that clicks on and off.

FIGS. 24A and 24B illustrate one variation of a fixed curve sheath having one or more conductive rings around or partially around the circumference of the sheath distal end for use with the end effector.

FIGS. 24C and 24D illustrate a straight, non-steerable sheath having one or more conductive rings around or partially around the circumference of the sheath distal end for use with the end effector.

FIGS. 24E and 24F illustrate a steerable sheath having one or more conductive rings around or partially around the circumference of the sheath distal end for use with the end effector.

### DETAILED DESCRIPTION

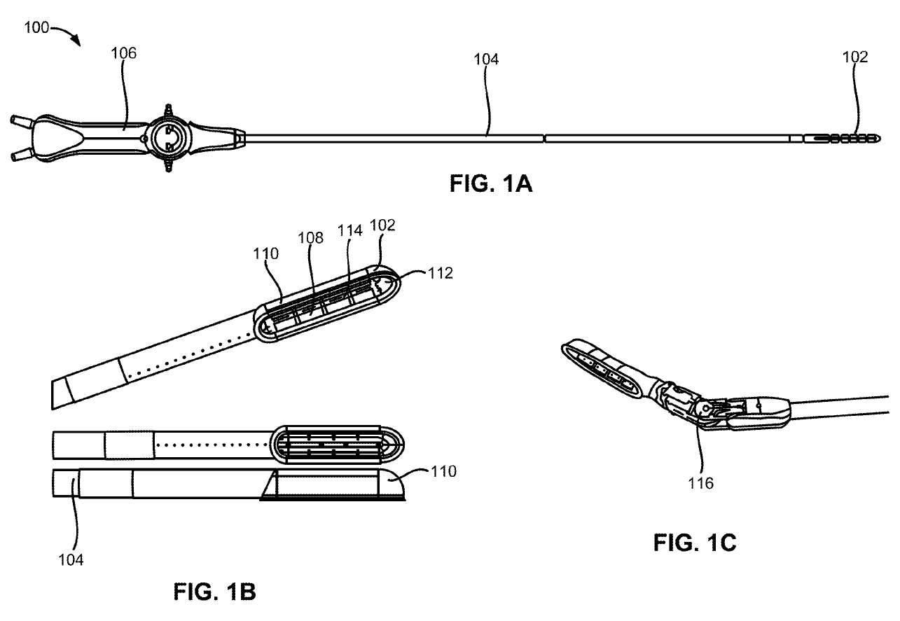

The present disclosure includes various electrosurgical devices, including ablation devices. FIG. 1A illustrates an example ablation device 100, which provides context for various alternative embodiments and optional features described herein. Unless otherwise indicated, any component, feature, method, etc. described herein may be utilized, alone or in any combination, in connection with an ablation device generally similar to ablation device 100. Ablation device 100 can have an end effector 102 connected to a distal end of an elongated shaft 104 and a handle 106 connected to a proximal end of the elongated shaft 104. The elongated shaft 104 can be straight and substantially rigid. However, flexible, curved, malleable, articulated, or other shafts could also be used depending on a variety of considerations.

Features of the present invention may incorporate features of treatment or ablation systems such as those described in U.S. Patent Application No. 18/227,632 filed July 28, 2023, the content of which is incorporated herein by reference.

Generally, any handle described herein with reference to any exemplary embodiment may be configured to be grasped by a human user (e.g., surgeon) and/or engaged by a non-human, mechanical and/or robotic device (e.g., a surgical robot). More generally, any handle described herein may comprise any structure that may be configured to be secured, held, and/or manipulated to position and/or restrain a PFA device, regardless of whether it may be held by a human (e.g., surgeon or assistant), robot, mechanical device, etc.

A connecting element, such as a cable (not shown), can be coupled to the handle 106 for connecting to devices such as power sources (e.g., external ablation energy sources). The end effector 102 can extend along a longitudinal axis of the device 100 and can have a total width of about 26mm. The end effector 102 can include a tissue engagement portion or working surface. As used herein, “working surface” may refer to a surface that is configured to come into contact with a target tissue of a patient. A working surface may include one or more individual surfaces, which may be contiguous or separated, and may include surfaces that are in any shape (planar, curved, concave, convex, etc.).

As will be discussed in more detail further herein, the working surface can comprise one or more insulators (or insulator portions) and one or more electrodes 108, which can be capable of being energized with electrical ablation energy, such as with bipolar RF ablation (RFA) energy or pulsed field ablation (PFA) energy. As used herein, “electrode” may refer to an element configured to deliver electrical energy to a target tissue through contact with the target tissue. Each electrode 108 can include a smooth surface area for contacting tissue. A variety of different metals or low electrical resistance materials can be used for the electrodes that are sufficiently electrically conductive to transfer potential and current to the tissue via ionic current density. For example, the electrodes 108 can be made of copper, nickel, gold, stainless steel, platinum, platinum-iridium, titanium, tin, metal on Kapton polyimide, metal on silicone or other elastomer, polymer-metal composites, hydrogels, or combinations thereof.

FIG. 1B illustrates close-up views of the end effector 102. The end effector 102 can comprise a housing 110 at a distal end of the elongated shaft 104. The housing 110 can comprise a blunt distal end and an interior cavity 112 so as to retain a flexible electrode array 114 which resides in the housing 110. The interior cavity 112 can receive tissue to be ablated during use via a vacuum source coupled to the end effector.

FIG. 1C illustrates one variation of the end effector 102 comprising a pivot joint 116 between the elongated shaft 104 and the end effector 102. The pivot joint 116 can be articulated via cables extending through the elongated shaft 104 to rotate the end effector 102 during use. It should be understood that the pivot joint 116 can be configured to rotate the end effector 102 in multiple different planes.

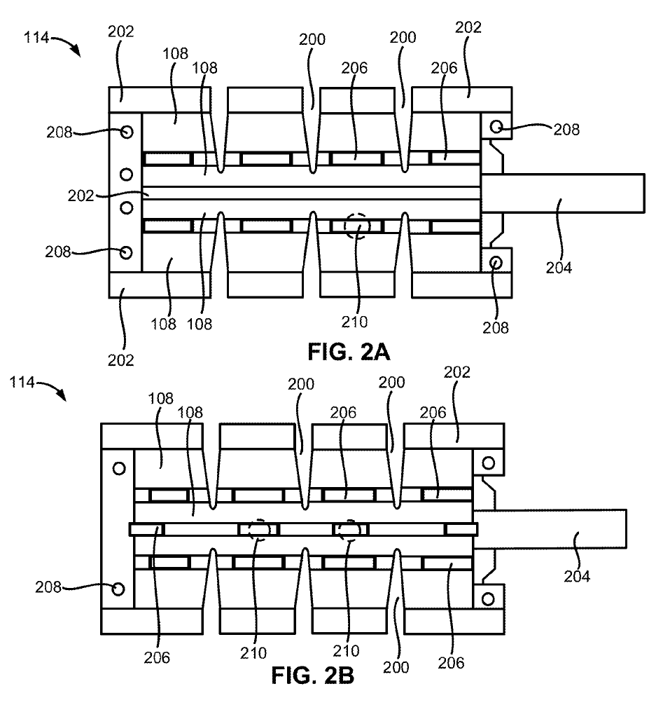

FIGS. 2A and 2B illustrate variations of the flexible electrode array comprising a plurality of electrodes 108 and a plurality of slits 200. The electrodes 108 can be spaced apart laterally along the flexible electrode array 114. In these variations, a total of four electrodes can be spaced apart, however, it should be understood that the flexible electrode array 114 can be configured with additional or fewer electrodes 108. The electrodes 108 can be electrically isolated from each other.

The flexible electrode array 114 can comprise an insulative portion 202 surrounding the electrodes 108 to spread heat and minimize hot spots. The flexible electrode array 114 can also comprise a tail 204 containing leads and electrical connection pads for communication with an energy source that provides energy to the electrodes 108.

The plurality of lateral slits 200 can comprise one or more pairs of lateral slits 200 extending at opposite sides of the flexible electrode array 114, as seen in FIGS. 2A and 2B. The lateral slits 200 can extend colinear from opposite sides of the flexible electrode array 114 across from each other to increase the flexibility of the flexible electrode array 114 in the plane of the slits 200.

The plurality of lateral slits 200 give the flexible electrode array 114 increased flexibility along the length of the end effector 102, enabling better conformability to cardiac curvature, and maintaining consistent contact throughout the ablation cycle.

In some variations, the one or more lateral slits 200 can be closed at a lateral edge of the flexible electrode array 114 (i.e., closed by the insulative portion 202).

In some variations, the flexible electrode array 114 can comprise no lateral slits.

The flexible electrode array 114 can also comprise axial slits 206 that extend in a longitudinal direction between the electrodes 108. In the variation of FIG. 2B, additional axial slits 206 can be positioned in the center of the flexible electrode array 114. The axial slits 206 can assist in bending the flexible electrode array 114 along the axial slit (e.g., about the longitudinal axis), increasing its flexibility in the plane of the axial slits.

In some variations, one or more circular cutouts 208 can be provided at the proximal end and the distal end of the end effector 102. The one or more circular cutouts 208 can be used for retention purposes for the flexible electrode array 114 to couple to the end effector 102 for stability (i.e., via a retention feature coupling the end effector with the flexible electrode array).

In some variations, the flexible electrode array 114 can have no slits or cutouts (i.e., the flexible electrode array 114 can be a solid element).

In some variations, a plurality of heat staking elements or equivalent mechanical features can extend from the end effector 102 and can be positioned at locations 210 of the one or more axial slits 206.

In some variations, the flexible electrode array 114 can be fixed within the end effector 102 and can be used independently of the end effector 102. The end effector 102 can be coupled to a vacuum source to draw tissue into the end effector 102 for subsequent ablation via the flexible electrode array 114.

The flexible electrode array 114 can be a single component with the ability to deliver bipolar energy. In some variations, the flexible electrode array 114 can be composed of a flex circuit or a printed circuit board (PCB). The flexible electrode array 114 can be composed of one or more layers of conductive electrode on a polyimide support with connecting vias and leads that can complete the circuit.

In some variations, the flexible electrode array 114 can further comprise a thermistor embedded within.

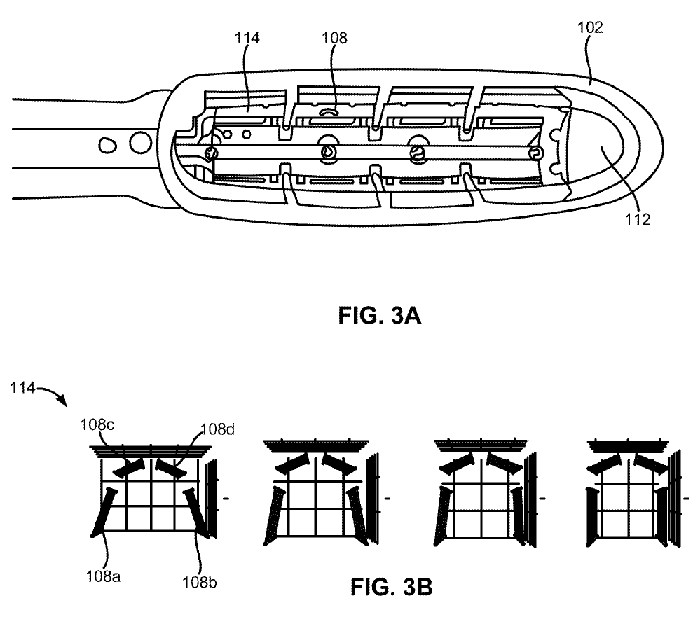

FIG. 3A illustrates the flexible electrode array 114 positioned within the end effector 102 according to one variation of the present invention. The end effector 102 can comprise a rounded housing 110 within an interior cavity 112 to hold the flexible electrode array. A vacuum source can be coupled to the end effector 102 to draw tissue into the interior cavity 112 to be treated by electrodes 108 of the flexible electrode array 114. The interior cavity 112 can have sufficient space within such that the flexible electrode array 114 can bend at various angles while the tissue is within the interior cavity.

FIG. 3B illustrates cross-sectional diagrams of the flexible electrode array 114 when bent at various angles. The outer electrodes 108a, b can be angled at about 70 (left most) to 90 degrees (right most) with respect to a target working surface. The inner electrodes 108c, d can be angled at about 30 to 40 degrees with respect to the target working surface. Such angle configurations provide a balance of ablation performance (depth and width of ablation while providing sufficient field strength to ablate), tissue suction performance, and tissue suction safety (i.e., reducing blood pool behind the tissue which can lead to thrombosis risk and stroke/clotting risk).

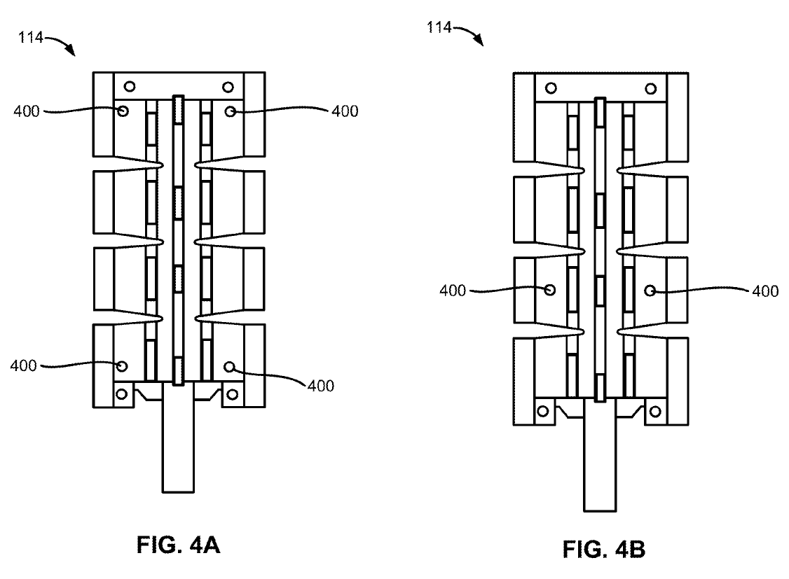

FIGS. 4A and 4B illustrate another variation of the flexible electrode array 114 having pacing or sensing electrodes 400. The sensing electrodes 400 can be distinct from the ablation electrodes 108 and can be embedded into the flexible electrode array 114 for the purpose of pacing/sensing cardiac signals (e.g., electrograms). As seen in the variations of FIGS. 4A and 4B, the sensing (or pacing) electrodes 400 can be placed along the ablation electrodes 108 at various locations along the ablation electrodes 108. The sensing electrodes 400 can comprise an insulative layer around a circumference of the sensing electrodes 400. One benefit of this variation is that the device 100 is provided with more focal pace, sense, and stimulation capability, thus providing improvements towards the overall signal quality and a more precise, targeted treatment at a specific tissue location.

The sensing electrodes 400 can be embedded within the flexible electrode array 114. The sensing electrodes 400 can comprise a diameter of less than 2mm to ensure that the sensing electrodes 400 do not significantly affect the resulting ablation. Accordingly, the ablation electrodes 108 on the flexible electrode array 114 can serve a dual purpose of ablation and pacing/sensing after tissue is pulled into the housing 110 via the vacuum source.

In some variations, the ablation electrodes can be multi-purpose electrodes (i.e., the ablation electrodes can also be pacing and sensing electrodes).

In some variations, a sensing source can be coupled to the electrodes 118, wherein the sensing source is configured to allow the electrodes to pace or sense electrical signals.

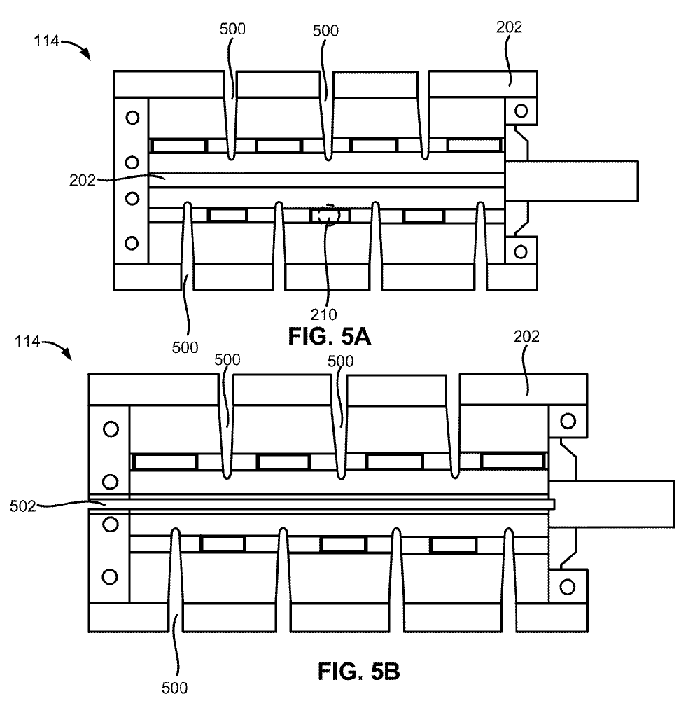

FIGS. 5A and 5B illustrate another variation of the flexible electrode array 114 having misaligned lateral slits 500. The flexible electrode array 114 can have a misaligned slit pattern to increase flexibility while mitigating the risk of lesion gaps. In this variation, individual slits are misaligned (i.e., not colinear) with each other in a lateral direction transverse to the longitudinal axis of the flexible electrode array 114. This variation lessens the possibility of gaps within the lesion along the length of the pod by staggering the lateral slits 500 while providing ablation flexibility. FIG. 5A illustrates a variation without a centerline slit (i.e., an insulative portion is in place at the centerline of the flexible electrode array).

FIG. 5B illustrates a variation with a centerline slit 502 to increase flexibility in a secondary plane and thus conformity to cardiac tissue. In this variation, the insulative portion at the base (i.e., at the proximal end) of the flexible electrode array 114 stabilizes the flexible electrode array 114.

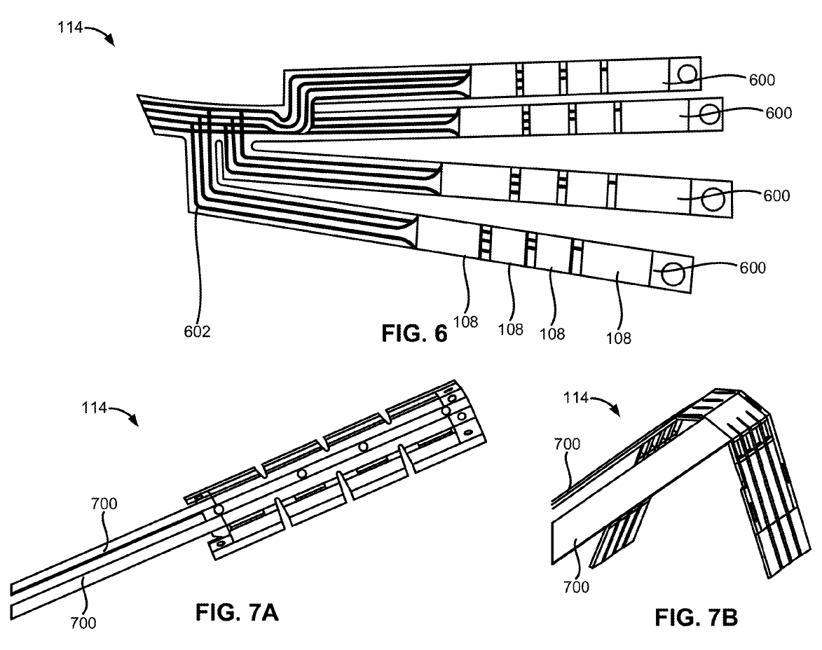

FIG. 6 illustrates a variation of the flexible electrode array 114 comprising one or more flexible arms 600 each connected to one or more electrical traces 602. Each flexible arm 600 can comprise one or more electrodes 108 which can be connected to an energy source via insulated electrical traces 602. The flexible arms 600 can be collapsible or expandable within the end effector 102. Since the flexible arms 600 can be collapsed or expanded more freely, the flexible electrode array 114 can treat different target sites with more versatility without compromising the ablation area. Accordingly, this variation also promotes added flexibility to conform to cardiac curvature.

The flexible arms 600 can collapse or expand independently such that only one or more flexible arms 600 extend radially from a collapsed position. The electrodes 108 can be spaced across each of the electrode arms and can comprise varying sizes along the arms 600. The electrical traces 602 can extend through each of the electrodes 108.

FIG. 7A illustrates a variation of the flexible electrode array 114 comprising two or more tails 700. The two or more tails 700 can each contain electrical traces and can connect the electrodes 108 of the flexible electrode array 114 to soldering pads and/or connectors (not shown). In this variation, the tails 700 can provide for a vertical orientation as the flexible electrode array 114 has two points at its proximal end at which it can bend laterally, as seen in FIG. 7B. The lateral bending provides added flexibility and versatility such that the flexible electrode array 114 can fit through lumens of different geometries.

In some variations, the flexible electrode array 114 can comprise two or more tails to alter the overall mechanical function of the flexible electrode array 114.

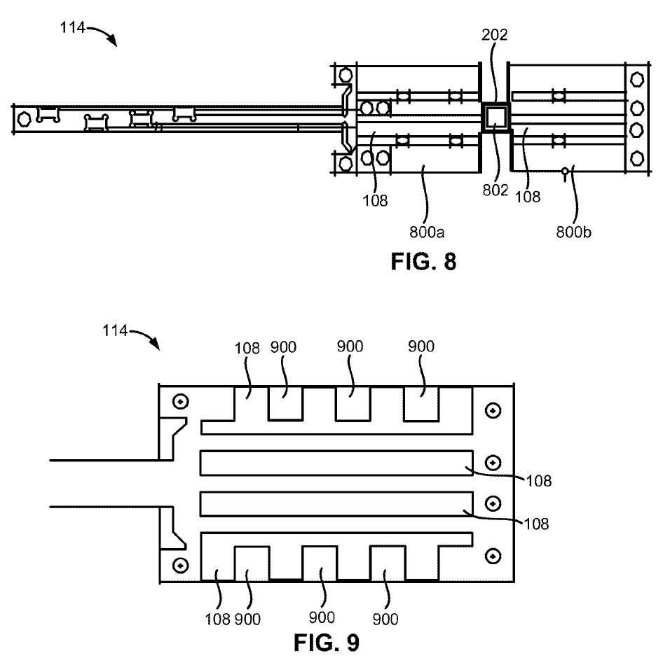

FIG. 8 illustrates a variation of the flexible electrode array 114 comprising two distinct pads 800a, b comprising distinct electrode areas. In this variation, a proximal pad 800a and a distal pad 800b can be connected via a bridge 802 which is narrower than the pads 800a, b. One or more electrical traces can run through the bridge 802, which is insulated accordingly.

This bridge 802 can increase flexibility along the length of the end effector 102, enabling better conformability to cardiac curvature, and maintaining consistent contact throughout the ablation cycle. This variation can also result in added retention of tissue in the end effector 102 as the narrow bridge 802 can provide more space for tissue to be drawn into the flexible electrode array 114.

FIG. 9 illustrates a variation of the flexible electrode array 114 comprising outside electrodes having a pattern of block-shaped cutouts 900 to increase flexibility in one plane. The cutouts 900 in this variation can help to reduce the risk of lesion gaps. The electrodes 108 in this variation can be continuous within one layer of the flexible electrode array 114.

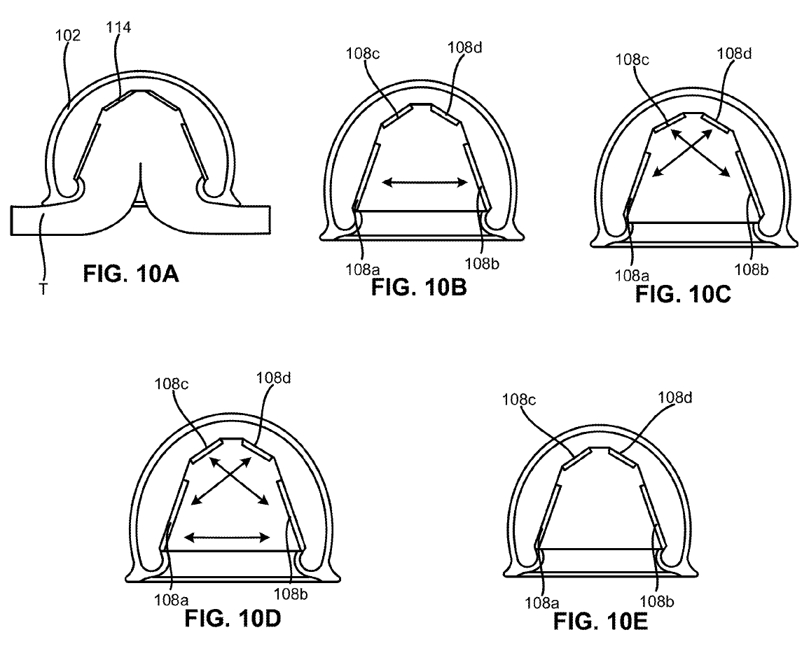

In some variations, the flexible electrode array 114 when combined with the end effector 102 can ablate a wide section of tissue (e.g., about 3mm thick to about 5mm thick). As seen in FIG. 10A, the tissue T can partially fold when pulled into the end effector 102, which results in an ablation width of about 6mm to about 10mm (e.g., about 8mm).

In some variations, the flexible electrode array 114 can be configured to fit within a trocar of about 6mm to about 10mm in inner diameter (e.g., about 8mm).

In some variations, the flexible electrode array 114 can be used in pulsed field ablation operations ranging from about 500V to about 5000V and having a duration of less than a millisecond. In some variations, the flexible electrode array can be used in radiofrequency ablation operations ranging from about 10V to about 100V and from about 400kHz to about 500kHz and having a duration of ten seconds or more. The flexible electrode array 114 can have sufficient separation between its electrical connectors and appropriate insulation features in order to withstand the applied voltage. The pulsed field ablation or radiofrequency ablation energy sources can be applied in series or in parallel.

FIGS. 10B to 10E illustrate various configurations of energy application to the tissue via the electrodes. The PFA or RFA energy can be delivered in various manners across an array of electrodes 108 via the flexible electrode array 114 without changing the physical nature of the flexible electrode array 114 or the end effector 102. The one or more electrodes 108 (e.g., 108a, b, c, d) allow versatility in the energy delivery options.

FIG. 10B illustrates that bipolar energy can be delivered to the tissue. In this variation, about eight bursts can be delivered between the outer electrodes 108a, b while inner electrodes 108c, d is not activated.

FIG. 10C illustrates a “multiplex” configuration in which bursts can be delivered diagonally between an outer electrode 108a, 108b and an inner electrode 108d, 108c, respectively. Delivery between each pair of outer and inner electrodes can be alternated such that each pair delivers energy about eight times.

FIG. 10D illustrates a “trifecta” configuration in which the “multiplex” delivery of FIG. 10C is performed, followed by the bipolar pattern in FIG. 10B.

FIG. 10E illustrates an “even” configuration in which a voltage divider evens the energy delivered across the four electrodes 108. The energy can be delivered simultaneously to each electrode 108 in this variation.

PFA lesions are often not immediately visible, making the process of creating a lesion set difficult. In some variations, having a device that can deliver both energies enables users to create a surface lesion to serve the purpose of denoting where the ablation was placed. In some variations, the radiofrequency energy can be used after PFA energy to mark where the PFA ablation took place. In other variations, the RFA energy can be used before the PFA energy is applied.

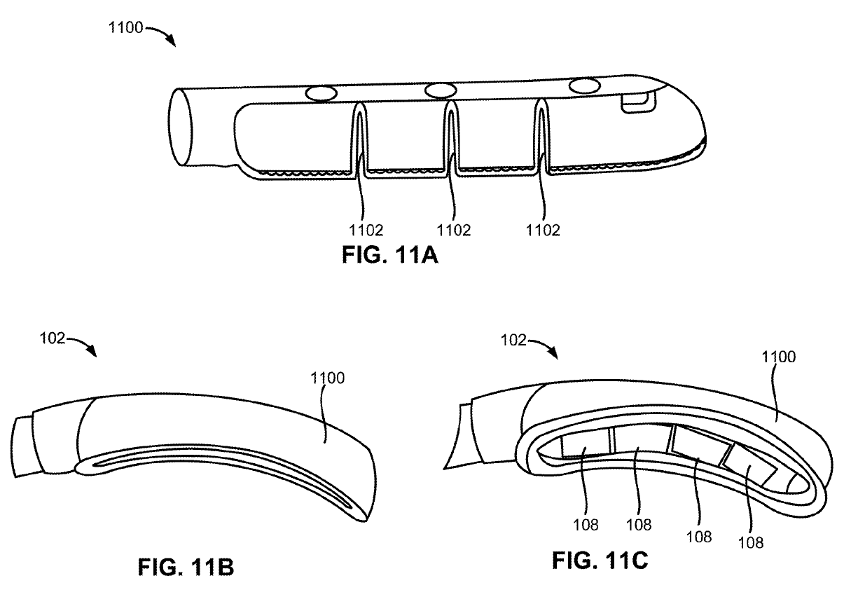

FIG. 11A illustrates one variation of an end effector insert 1100 having one or more slits 1102. The one or more slits 1102 can be one or more pairs of lateral slits 1102 extending at opposite sides of the end effector 102. The one or more slits 1102 can extend from opposite sides of the end effector 102 across from each other symmetrically to increase the flexibility of the end effector 102 in the plane of the one or more slits 1102. The end effector 102 can comprise a housing 110 and an interior cavity 112 in which a vacuum source can be applied thereto to draw tissue within.

FIGS. 11B and 11C illustrate the end effector 102 bending with the flexible electrode array 114 positioned within the end effector 102. The end effector 102 can both be flexible and compressible without collapsing or sacrificing the structural integrity of the flexible electrode array 114 or the end effector 102. The flexibility of the end effector 102 increases the conformability of the end effector 102 against a curved cardiac surface. Further, the compressibility of the end effector 102 can allow the end effector 102 to fit through a catheter shaft during delivery.

In some variations, the one or more slits 1100 can be altered to change the flexibility of the end effector 102. For example, the direction (e.g., axial or lateral), size (e.g., width or depth), durometer, and/or pattern (e.g., symmetrical or asymmetrical) of the one or more slits 1100 can be changed in alternate variations.

The end effector 102 and its components can be made of elastomer such as thermoplastic polyurethane, silicone, thermoplastic elastomer vulcanizate, or other elastomer, although other materials can be used such as nylon, polycarbonate, other plastic, insulated metal, or a combination thereof.

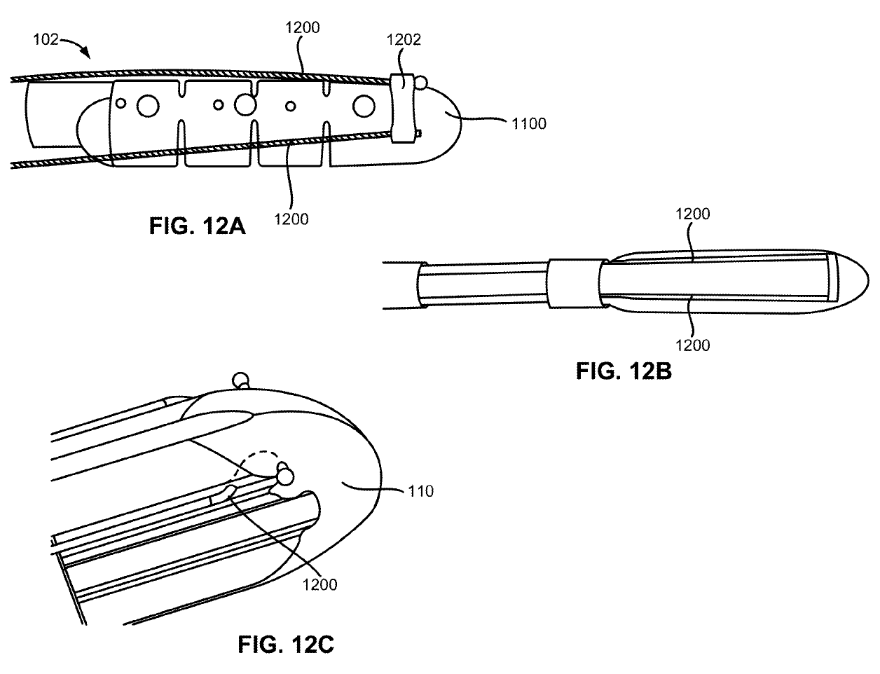

FIGS. 12A to 12C illustrate a variation of the end effector 102 comprising a plurality of articulation bands 1200. The end effector 102 can comprise the insert, an outer sleeve, and the plurality of articulation bands 1200 therebetween. The articulation bands 1200 can be attached or anchored at a distal tip of the end effector 102 and can be used to control the end effector 102 via the handle. The bands 1200 can be attached at an attachment portion 1202 on insert 1100. The outer sleeve can be made of silicone or another elastomer.

The articulation bands 1200 can provide for user control especially when the device is under the weight of the heart or under tension of the pericardium as the pericardium presents external forces that can make the end effector 102 challenging to articulate and control. To this end, the elongated shaft 104 can be steerable flexible catheter or a steerable rigid shaft platform. Steering can be in one or more planes via the articulation bands 1200.

As seen in FIG. 12C, the bands 1200 can comprise a wire component that is weaved through the housing of the end effector 102 and anchored at the distal tip of the end effector 102. This provides the user with control over the end effector 102 while providing a strain relief effect to the device.

In some variations, the articulation bands 1200 can comprise different wire types, size, materials, or termination style (e.g., weaving, termination component).

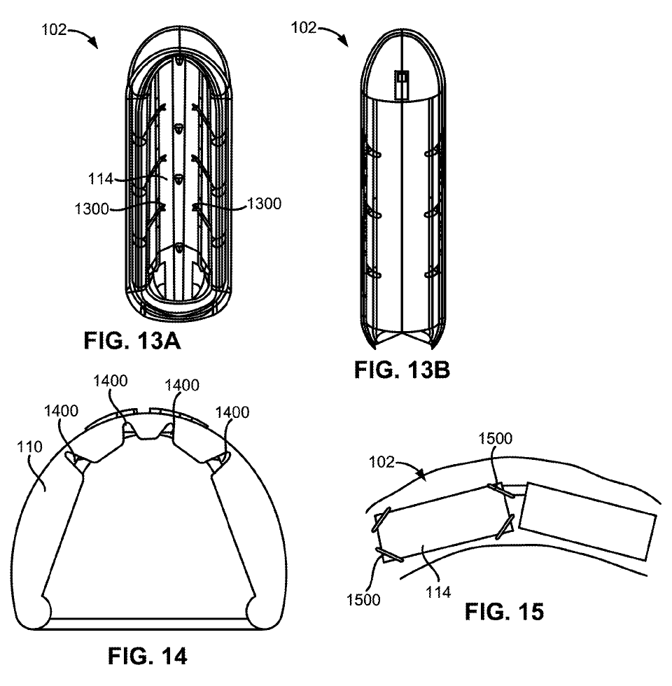

FIGS. 13A and 13B illustrate a variation of the end effector 102 comprising retention features for placing the flexible electrode array 114 within the housing 110. In this variation, the end effector 102 can comprise a plurality of heat stakes 1300 extending into the interior cavity of the end effector 102. The heat stakes 1300 can couple to circular cutouts 208 (see FIGS. 2A and 2B) in the flexible electrode array 114 for holding the flexible electrode array 114 within the end effector 102 in a predetermined location. This contributes to the consistency of electrode spacing, which can be predetermined.

In some variations, the heat stakes 1300 can have different geometries (i.e., different shapes, sizes, etc.), quantities, or patterns/locations. In other variations, a press fit, and/or ultrasonic welding can be used to engage flexible electrode array 114 with end effector 102. In other variations, mechanical or chemical adhesion may be achieved through overmolding, solvent bonding, pressure-sensitive adhesives, permanent adhesives, or other mechanisms.

FIG. 14 illustrates a variation of the end effector 102 comprising one or more compression notches 1400 on an inside of the housing 110. The compression notches 1400 can decrease a wall thickness of the housing 110 in locations beneficial for the end effector 102 to fold. This increases the flexibility and the compressibility of the device, making the device compatible with smaller delivery devices. The notches 1400 can be spread apart radially along an underside of the housing 110 and can extend longitudinally along the housing 110.

FIG. 15 illustrates a cross-sectional view of one variation of the end effector 102 and the flexible electrode array 114 in which the housing 110 of the end effector comprises one or more cutouts or flaps 1500 that are used as corner brackets for retaining the flexible electrode array 114 in a predetermined location. The flexible electrode array 114 can snap or otherwise lock into the cutouts or flaps 1500 to couple to the end effector 102.

In some variations, the end effector 102 can comprise a plurality of mechanical interlocks generated during overmolding configured to retain the flexible electrode array 114 within the housing 110.

In some variations, the end effector 102 can comprise an adhesive layer configured to retain the flexible electrode array 114 within the housing 110.

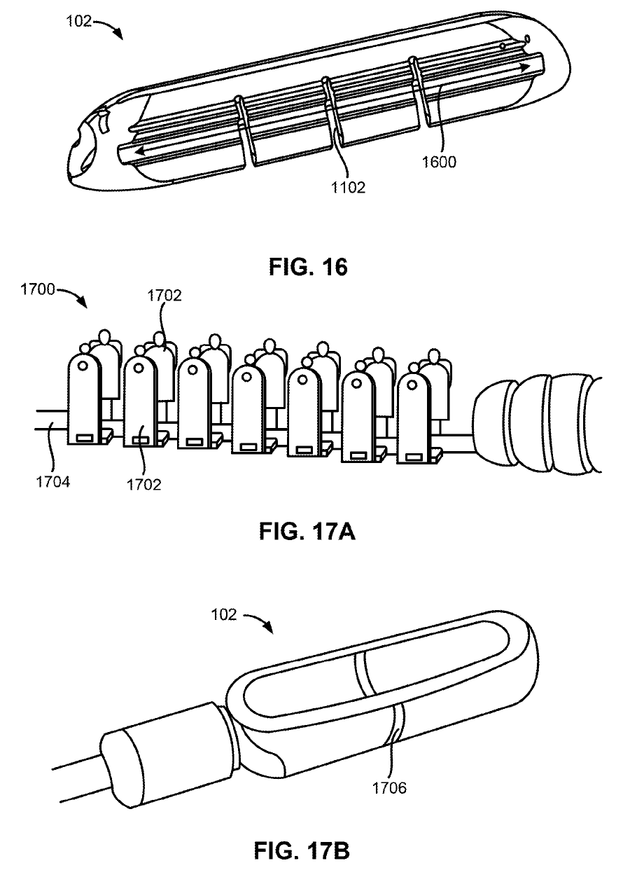

FIG. 16 illustrates a variation of the end effector 102 comprising one or more slits 1100 and a channel 1600 that runs along the length of the end effector 102. The channel 1600 can run longitudinally and allows for tissue fluid to travel between layers of the end effector 102 for eventual removal. This allows clearance of the fluid during use as the end effector 102 layers are tightly fit to one another such that fluid can sometimes become trapped in between, leading to build-up, poor seal, and potentially electrical interference. In some variations, multiple channels 1600 can be provided within the end effector 102.

FIGS. 17A and 17B illustrate variations of an insert 1700 for use within the end effector 102. The insert 1700 can comprise flexible ribs 1702 so as to balance rigidity and compressibility of the end effector 102. FIG. 17A illustrates an insert 1700 with inter-connected tabs or ribs 1702 that thread onto a central shaft 1704 and flex individually. There can be about ten to twelve individual flexible ribs 1702 that allow the end effector 102 to fold in half but not collapse. In some variations, springs, ribs, or hinges can be threaded onto the central shaft 1704.

FIG. 17B illustrates wherein an insert that is a metal element (e.g., super-elastic nitinol wire or sheet) comprising U-shaped rib 1706 that can strengthen the end effector 102. FIG. 17B illustrates a single band; however, in some variations, there can be configurations with one or more bands along the length of the end effector 102.

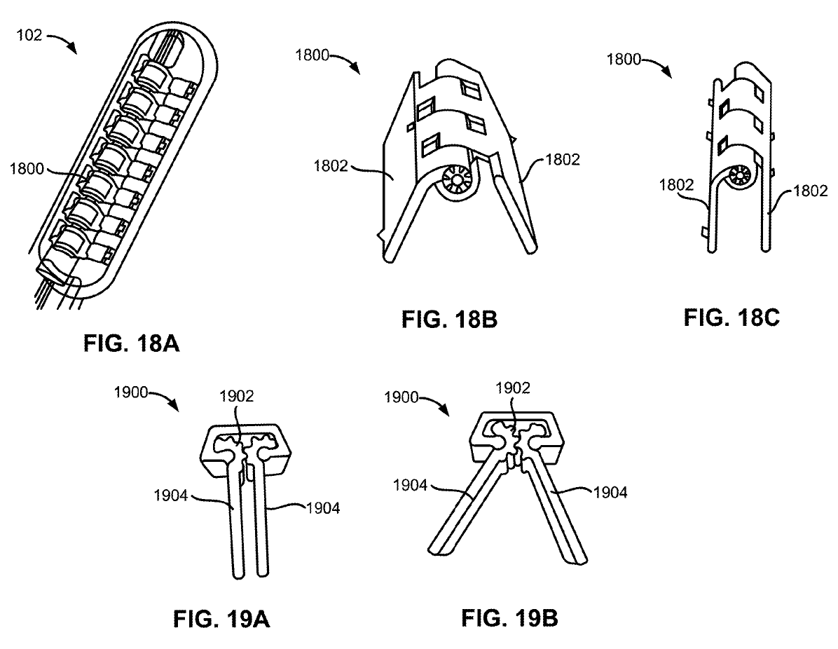

FIGS. 18A to 18C illustrate one variation of the insert 1800 for use within the end effector 102 comprising a hinge mechanism. The insert 1800 can be made of components that thread onto a central shaft that allow opposite portions to hinge apart (as seen in FIG. 18B) or together (as seen in FIG. 18C). This allows the end effector 102 to be more compressible for use with smaller delivery devices.

In some variations, the insert 1800 can create a flexible or rigid flat section for electrode placement. In other variations, the insert 1800 can have individual rib-like features that hold the electrodes 108. The insert 1800 can be used in place of the flexible electrode array 114 or in combination therewith.

FIGS. 19A and 19B illustrate one variation of the insert 1900 for use within the end effector 102 comprising a continuous gear 1902. The continuous gear 1902 can be connected to a torque wire (not shown) to collapse and expand the end effector 102. The continuous gear 1902 can comprise legs 1940. This allows the end effector 102 to be compatible with smaller delivery devices. This configuration can also allow a user to control the electrode geometry by changing the angle of the electrode with respect to the tissue and gives the user the ability to 'pinch' tissue to reduce the captured blood pool.

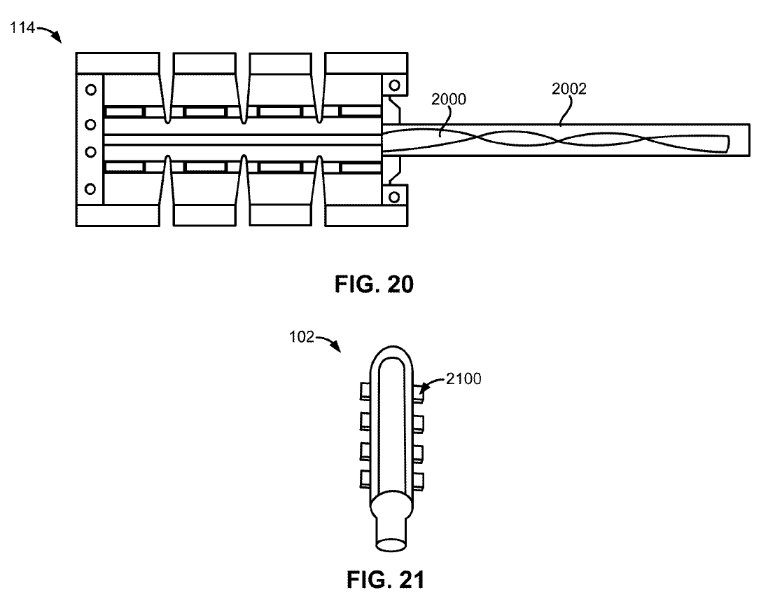

FIG. 20 illustrates a variation of the flexible electrode array 114 comprising a tail 2000 in a helical configuration within a vacuum lumen 2002. The tail 2000 can be helically shaped such that it can only partially occupy space in the vacuum lumen 2002, preventing occlusion of the vacuum lumen 2002. This allows the flexible electrode array 114 to be positioned at the end of the vacuum lumen 2002 without the need for another lumen to hold the tail 2000. Accordingly, the flexible electrode array 114 can exit the vacuum lumen 2002 without potentially disrupting other lumens within the device.

One technical problem encountered by the inventors is how to route the tail of the flexible electrode array 114 down a lumen (e.g., a vacuum lumen) such that there is no need for a new lumen (which affects manufacturability) or occlusion of the vacuum lumen. To this end, a straight cut flexible electrode array 114 can disrupt vacuum flow in the lumen due to bunching/kinking, affecting vacuum efficacy negatively. One technical solution discovered by the inventors is to introduce a helical tail configuration within the lumen. To position the tail 2000 within the vacuum lumen 2002, the tail 2000 of the flexible electrode array 114 can be wrapped around a cylinder. Traces of the flexible electrode array 114 can hold the helical shape, allowing the flexible electrode array 114 to be placed into the lumen and follow a path down the surface of the lumen limiting the occlusion caused by the flexible electrode array 114 potentially interrupting flow.

The helical configuration of the tail 2000 can comprise an elastic material such that the elongated shaft 104 in which the vacuum lumen resides can experience added flexibility or a spring-like function to the elongated shaft 104.

FIG. 21 illustrates the end effector 102 having one or more external heat stakes 2100. The external heat stakes 2100 can be placed on an outer surface of the end effector 102 and can vary in size, shape, and placement. The heat stakes 2100 can comprise various positions (e.g., at the edge, top, or within the end effector 102) or shapes (e.g., circular, elliptical, rectangular, etc.). The heat stakes 2100 can be spaced apart longitudinally along the end effector 102.

The external heat stakes 2100 provides for no interference with the electrodes 108 of the flexible electrode array 114.

One technical problem encountered by the inventors is how to provide the end effector 102 with heat stakes 2100 with precision, how to prevent silicone unfurling, and how to prevent interference of press and stakes within the flexible electrode array 114. One technical solution discovered by the inventors is to relocate the internal heat stakes meant to retain the flexible electrode array 114 to the exterior edge of the end effector 102. The external heat stakes 2100 can allow for stakes to travel through all components of the end effector 102 to secure them together. The external heat stakes 2100 position and size can be optimized to improve the mechanical characteristics of the end effector 102 to limit/prevent silicone unfurling, improve circuit retention, and improve rigidity. Additionally, the external heat stakes 2100 can be manufactured along with the end effector 102 via a single joining method.

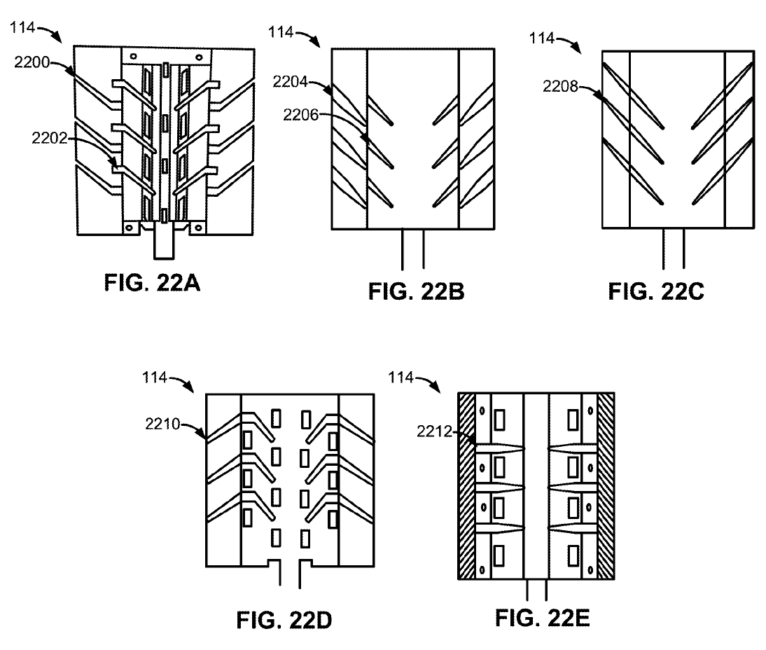

FIG. 22A to 22E illustrate variations of the flexible electrode array 114 having one or more creases 2200 to improve rigidity, improve fit of the flexible electrode array 114 in the end effector 102, and to allow for external heat staking as described herein.

In this variation, the flexible electrode array 114 can be scored, perforated, and/or heat creased to create crease lines within the flexible electrode array 114. The flexible electrode array 114 can be folded at specific linear points to transition the flexible electrode array 114 from a flat structure to a natural 3D structure that can conform structurally when seated in the end effector 102.

In other variations, the creases can be made with other methods, including alternative methods of creasing, heat and fold techniques, scoring with a laser cutter, or perforation with a laser cutter.

FIG. 22A illustrates a variation with creases 2200 extending proximally at an angle from an edge of the flexible electrode array 114, with a portion that is perpendicular to a longitudinal axis of the flexible electrode array 114. Creases 2202 can also be formed from within the flexible electrode array 114.

FIG. 22B illustrates a variation with creases 2204 extending proximally at an angle from an edge of the flexible electrode array 114. Creases 2206 can also be formed from within the flexible electrode array 114 at the same angle. FIG. 22C illustrates a variation with creases 2208 extending proximally at an angle from an edge of the flexible electrode array 114 further towards the centerline of the flexible electrode array 114 than the creases 2204 in FIG. 22B.

FIG. 22D illustrates a variation with creases 2210 extending from an edge of the flexible electrode array 114 in a “V-shape” with an elongated middle portion extending radially. FIG. 22E illustrates a variation with creases 2210 extending from within the flexible electrode array 114 radially.

In addition to creasing, the flexible electrode array 114 can be wrapped around the edge of the end effector 102, in order to provide increased structural integrity of the flexible electrode array 114 and the device as a whole. This wrapped structure can also allow for external heat stakes on the end effector 102 as the external heat stakes can extend through creases in the flexible electrode array 114, nesting within the creases for stability.

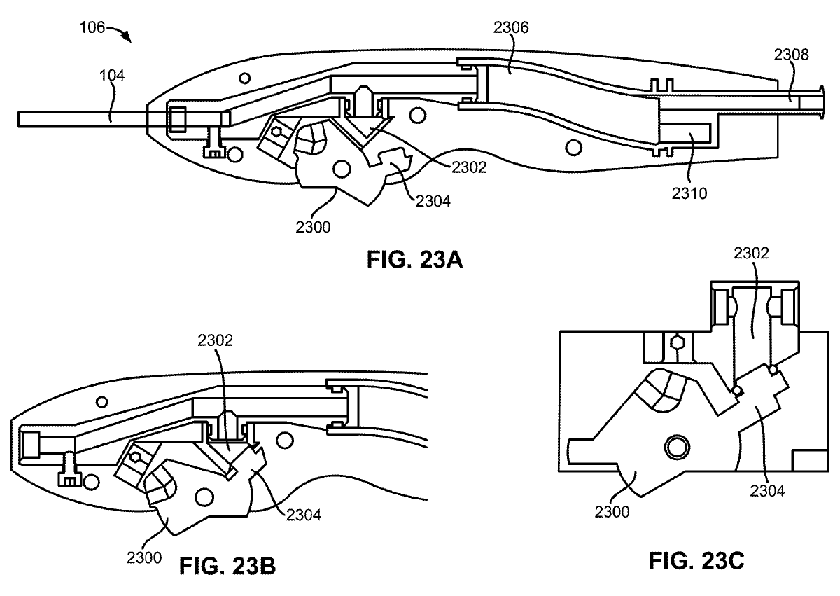

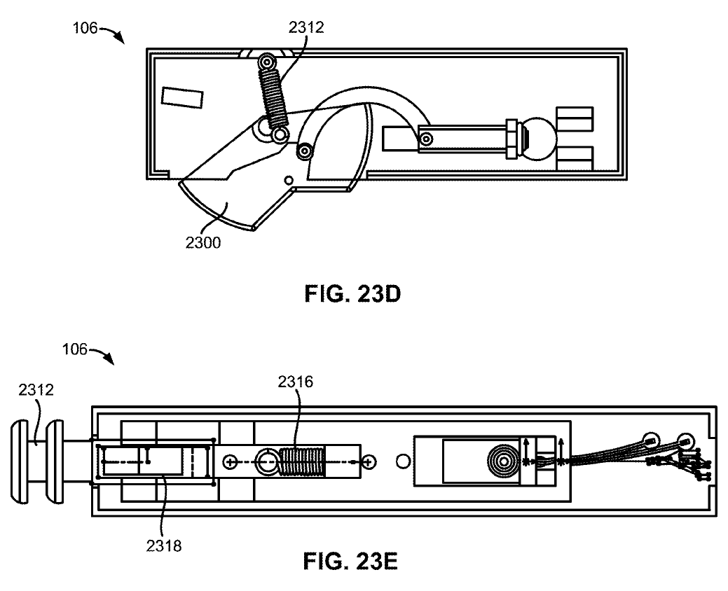

FIG. 23A illustrates a handle 106 with an integrated vacuum switch 2300 for use with the device 100. The handle 106 can comprise a switch 2300 and a chamber 2302 on an underside thereof. The switch 2300 can be a rocker toggler switch that can rotate with respect to the handle 106 when a user applies pressure to the switch 2300. The switch 2300 can comprise a protrusion 2304 that couples with the chamber 2302 to either close or open the chamber 2302 based on the position of the switch 2300. The switch 2300 can comprise a compression spring element within to transition the switch 2300 between positions.

In the closed position of FIG. 23B, vacuum is applied to the end effector 102. In the open position of FIG. 23A, vacuum is not applied to the end effector 102, as the chamber 2302 draws in ambient air (i.e., from within the handle).

The chamber 2302 can allow the user to engage or disengage the vacuum source directly by toggling the switch 2300 between an open and closed position. This allows for the user to control the vacuum with one hand, improving the ergonomics of the device. To this end, the chamber 2302 can be connected to the vacuum line 2306 extending through the handle, which is in turn connected to the vacuum source (not shown) via a luer fitting 2308 at a proximal end of the handle 106.

In some variations, an electrical wire connection 2310 can also be provided at the proximal end of the handle 106 for connection to a cable to power the electrodes.

The vacuum can extend through the elongated shaft 104 and to the end effector 102 for suction during use.

One technical problem encountered by the inventors is how to increase the ergonomics of the device, as some devices require that the vacuum must be activated or deactivated via a stopcock, meaning that either the user has to let go of the device with one hand or have another user run the stopcock. One technical solution discovered by the inventors is to provide an accessible switch on the handle to improve ergonomics and efficiency.

FIG. 23C illustrates a close-up view of the switch 2300 which can comprise an O-ring pressed against a chamfered hole of the chamber 2302 to seal the chamber 2302.

FIG. 23D illustrates another variation of the handle 106 comprising a rocker switch 2300 and a tensile spring 2312. FIG. 23E illustrates another variation of the handle 106 comprising a pen-like clicking element 2314 that clicks on and off. The clicking element 2314 can be coupled to a spring 2316 that actuates the vacuum source on and off via sliding seals 2318.

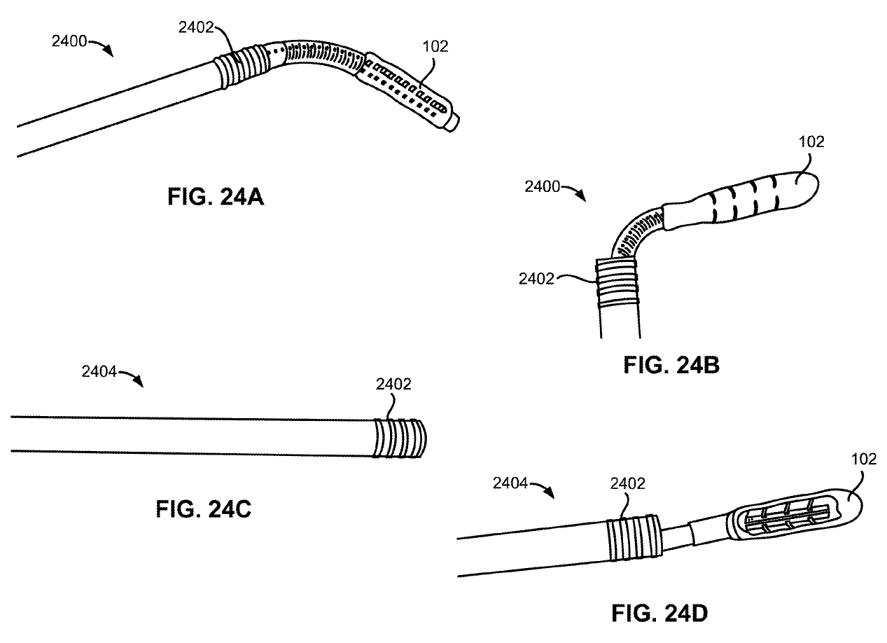

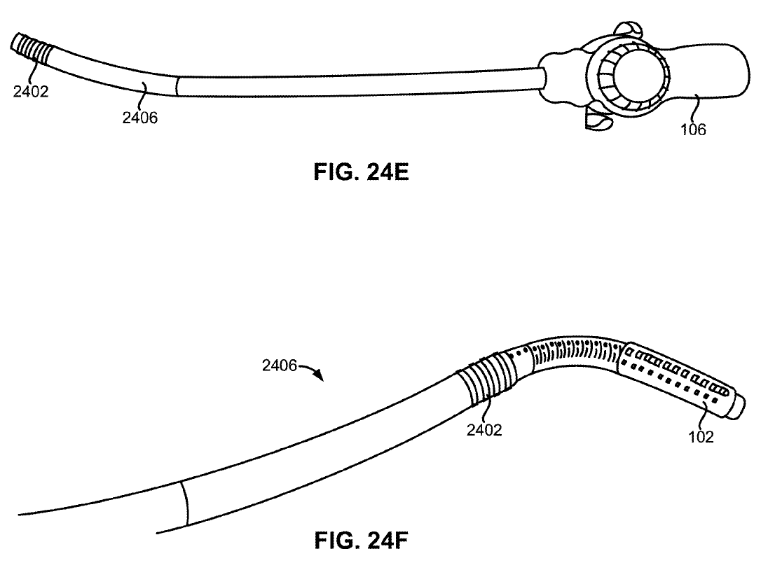

FIGS. 24A and 24B illustrate one variation of a fixed curve sheath 2400 having one or more conductive rings 2402 around or partially around the circumference of the sheath distal end for use with the end effector 102. Each conductive ring 2402 can be electrically isolated from each other and can be connected to a mapping system to enable pacing and/or sensing functionality. The mapping system can provide visibility and/or positioning for the device 100 distal end during use.

FIGS. 24C and 24D illustrate a straight, non-steerable sheath 2404 having one or more conductive rings 2402 around or partially around the circumference of the sheath distal end for use with the end effector 102. Each conductive ring 2402 can be electrically isolated from each other and can be connected to a mapping system to enable pacing and/or sensing functionality. The mapping system can provide visibility and/or positioning for the device 100 distal end during use. In some variations, the sheath 2404 inner diameter and outer diameter can be fixed.

FIGS. 24E and 24F illustrate a steerable sheath 2406 having one or more conductive rings 2402 around or partially around the circumference of the sheath distal end for use with the end effector 102. Each conductive ring 2402 can be electrically isolated from each other and can be connected to a mapping system to enable pacing and/or sensing functionality. The mapping system can provide visibility and/or positioning for the device 100 distal end during use. The sheath 2406 can comprise articulation bands that articulates the sheath in at least two different planes.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps or operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit, or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of”, when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially”, “about” and “approximately” as used herein mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0cm” can be interpreted to mean “1.0cm” or between “0.9cm and 1.1cm.” When terms of degree such as “about” or “approximately” are used to refer to numbers or values that are part of a range, the term can be used to modify both the minimum and maximum numbers or values.

It will be understood by one of ordinary skill in the art that the various methods disclosed herein may be embodied in a non-transitory readable medium, machine-readable medium, and/or a machine accessible medium comprising instructions compatible, readable, and/or executable by a processor or server processor of a machine, device, or computing device. The structures and modules in the figures may be shown as distinct and communicating with only a few specific structures and not others. The structures may be merged with each other, may perform overlapping functions, and may communicate with other structures not shown to be connected in the figures. Accordingly, the specification and/or drawings may be regarded in an illustrative rather than a restrictive sense.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

We claim:

1. An ablation device, the device comprising:
a handle;
an elongated shaft extending from the handle along a longitudinal axis;
a flexible electrode array at a distal end of the elongated shaft, wherein the flexible electrode array comprises a plurality of electrodes and an insulative portion, wherein each of the plurality of electrodes extend longitudinally along the flexible electrode array, one or more slits extending transverse to the longitudinal axis, the one or more slits configured to increase the flexibility along a length of the flexible electrode array; and
an end effector at the distal end of the elongated shaft, the end effector comprising a housing in which the flexible electrode array resides, wherein the housing is flexible and is configured to conform to a tissue of a patient.

2. The ablation device of claim 1, wherein the one or more slits comprises one or more pairs of slits extending laterally at opposite sides of the flexible electrode array.

3. The ablation device of claim 2, wherein individual slits of the one or more pairs of slits are colinear to each other in a lateral direction transverse to the longitudinal axis.

4. The ablation device of claim 2, wherein individual slits of the one or more pairs of slits are misaligned with each other in a lateral direction transverse to the longitudinal axis.

5. The ablation device of claim 1, wherein the plurality of electrodes comprises one or more ablation electrodes and one or more sensing electrodes.

6. The ablation device of claim 1, wherein the flexible electrode array comprises a center slit extending longitudinally through a center of the flexible electrode array.

7. The ablation device of claim 1, wherein the flexible electrode array comprises a plurality of arms, wherein the plurality of arms are collapsible within the end effector.

8. The ablation device of claim 1, wherein the flexible electrode array further comprises a tail comprising one or more electrical traces connected to the plurality of electrodes.

9. The ablation device of claim 1, wherein the flexible electrode array comprises a plurality of cutouts configured to bend the flexible electrode array.

10. The ablation device of claim 1, further comprising a suction source coupled to the end effector, the suction source configured to apply suction to the end effector to draw tissue into the housing.

11. The ablation device of claim 1, further comprising an energy source coupled to the plurality of electrodes, the energy source configured to provide energy to the plurality of electrodes.

12. The ablation device of claim 1, further comprising a sensing source coupled to the plurality of electrodes, the sensing source configured to allow the electrodes to pace or sense electrical signals.

13. An ablation device, the device comprising:
an elongated shaft along a longitudinal axis;
a flexible electrode array at a distal end of the elongated shaft, wherein the flexible electrode array comprises a plurality of electrodes and an insulative portion; and
an end effector at the distal end of the elongated shaft, the end effector comprising a housing in which the flexible electrode array is anchored, wherein the housing is flexible to conform to a tissue of a patient.

14. The ablation device of claim 13, wherein the end effector comprises one or more cutouts.

15. The ablation device of claim 13, further comprising a plurality of articulation bands attached to a distal tip of the end effector.

16. The ablation device of claim 13, further comprising a plurality of heat stakes coupled to the end effector and configured to retain the flexible electrode array within the housing.

17. The ablation device of claim 13, further comprising a plurality of notches within an inner surface of the housing.

18. The ablation device of claim 13, wherein the end effector comprises a channel extending along a length of the housing.

19. The ablation device of claim 13, wherein the end effector comprises one or more flexible ribs within the housing.

20. The ablation device of claim 13, wherein the end effector comprises one or more metallic stabilization elements within the housing.
